(19) United States
(12) Patent Application Publication
(10) Pub. No.: US 2024/0197367 A1
(43) Pub. Date: Jun. 20, 2024

(54) BONE SCREW AND SLEEVE REMOVER

(71) Applicant: Ulrich GmbH & Co. KG, Ulm (DE)

(72) Inventors: Stefan MIDDERHOFF, Ulm (DE); Michael FROMM, Ulm (DE); Frauke ZIMMERMANN, Ulm (DE)

(73) Assignee: Ulrich GmbH & Co. KG, Ulm (DE)

(21) Appl. No.: 18/286,604

(22) PCT Filed: May 10, 2021
(86) PCT No.: PCT/EP2021/062323

§ 371 Date: Oct. 12, 2023

(51) Int. Cl. A61B 17/70; A61B 17/88

(52) U.S. Cl. CPC A61B 17/702; A61B 17/7032; A61B 17/8863

(57) ABSTRACT

A bone screw has a threaded screw shank and a screw head that in turn has two opposite legs defining a receptacle between them and integrally connected to a tissue-protecting sleeve. The tissue-protecting sleeve has a slot that runs from the sleeve's one edge to the opposite edge. Further, a sleeve remover has a corpus structured as a hollow cylinder, which has a separation element at one end, on its inside circumference, which element extends over part of the circumference direction.

The invention relates to a bone screw having a screw shank that has a thread, and a screw head that has two legs that lie opposite one another and form a receptacle between them, which legs are connected, in one piece, with a tissue-protecting sleeve, wherein the tissue-protecting sleeve has a slot that runs from the one edge of the sleeve to the opposite edge. The invention furthermore relates to a sleeve remover.

Bone screws are widespread in medicine for surgical applications, and are used in the embodiment of pedicle screws for correction and stabilization of the spinal column. The screw shank, configured as a threaded shank, is supplemented by a screw head structured as a tulip head, having a receptacle formed between the legs, in which receptacle a rod can be placed and fixed in place from the cranial to the caudal direction, which rod connects adjacent vertebrae or entire groups of vertebrae with one another. Such pedicle screws are known, for example, from EP 2 452 642 A2. The placement of the bone screw with the rod can take place in a conventional manner, over an operation field that has been exposed over a large area, wherein, however, the musculature must be released from the spinous processes of the vertebrae and moved aside so as to expose the pedicles.

Therefore a minimally invasive access is more advantageous, in which a small skin incision is made above the location where the bone screw is to be placed. With the use of dilatation sleeves, an operation channel for setting the bone screw or pedicle screw is created. Furthermore a tissue-protecting sleeve is required, which is coupled with the bone screw and must be removed again after setting and fixation of the rod.

Therefore it is a task of the present invention to make available a bone screw having simplified intra-operative handling. It is furthermore a task to make available a sleeve remover for easy removal of the tissue-protecting sleeve.

This task is accomplished by means of a bone screw having the characteristics of claim 1 and by means of a sleeve remover having the characteristics of claim 12. Advantageous embodiments with practical further developments of the invention are indicated in the dependent claims.

The bone screw mentioned initially is characterized in that the tissue-protecting sleeve is firmly connected with the screw head, namely both legs, in other words no measures are required for releasably coupling the tissue-protecting sleeve with the bone screw before the operation, and releasing this coupling again intra-operatively. The bone screw together with the tissue-protecting sleeve can be handled as one piece, so that the tissue-protecting sleeve constantly and reliably performs its protective function for the operation channel. Nevertheless the tissue-protecting sleeve must be separated from the bone screw again after it has performed its function, and for this purpose the tissue-protecting sleeve has a particularly suitable configuration, having a slot that extends over its entire length expanse, which slot creates an access that can be used for the separation.

It is practical if, for this purpose, the legs of the screw head are connected with the tissue-protecting sleeve by way of a planned breaking point. This planned breaking point can be acted on for separation, proceeding from the slot.

Due to the slot, the tissue-protecting sleeve is not closed in the circumference direction, and as a result its stability is restricted. In order to balance this out or to at least partially compensate for it, it is provided that the tissue-protecting sleeve has a stabilization bracket on the edge that lies opposite the legs.

For simplified handling during the operation, the tissue-protecting sleeve has an opening on the side that lies opposite the slot, which opening extends from the edge facing the leg over part of the longitudinal expanse of the tissue-protecting sleeve, through which opening the rod can be introduced into the receptacle and laid in place, even when the tissue-protecting sleeve is still connected with the screw head.

It is furthermore preferred if the tissue-protecting sleeve is circular in cross-section and the slot extends over an arc having a length between 15° and 90°, in other words the arc covers an angle region that corresponds to a minimum of 15°. The usable interval for the size of the arc ranges up to 90°, in other words a quarter circle, so that sufficient material remains for the purpose of the tissue-protecting sleeve.

It is very particularly preferred if a sleeve remover that can be placed over the tissue-protecting sleeve is provided for interaction with the bone screw, which sleeve remover has a separation element on its end facing the legs, in the configuration when it is set onto the tissue-protecting sleeve, on its inside circumference, and the expanse of the separation element, which expanse lies in the circumference direction, is smaller than the expanse of the slot, which expanse lies in the circumference direction. The separation element can therefore be introduced into the slot and guided over the entire length of the tissue-protecting sleeve, to its end that faces the screw head, so that there it can act close to the screw head, in particular on the planned breaking point, and detach the tissue-protecting sleeve. For this purpose, the separation element is configured as a blade that is guided along the planned breaking point by means of rotating the sleeve remover about its longitudinal axis and cuts this point open. By means of the blade, a clean cut is ensured, at which no splinters or chips occur and also no burrs are formed. The cut edge, in particular on the screw head, is not formed with a sharp edge. It should also be noted that the blade acts on the planned breaking point in the direction of the longitudinal axis of the screw, in the direction of the interior, and thereby prevents the legs from being bent open.

For the introduction of the torque for rotating the sleeve remover, the sleeve remover has a drive opening in its circumference. A pin for the drive can be inserted into this drive opening.

It is practical if the drive opening is arranged on the end that faces away from the legs and/or on the side that lies opposite the separation element, because handling is improved in this manner.

According to the intended purpose, a rod can be placed into the receptacle between the legs, wherein the separation element is so far away from the edge of the sleeve remover that the separation element is assigned to the planned breaking point when the edge lies on the rod.

It serves to secure the rod in the receptacle that a thread for accommodating a fixation screw is formed on the inside of the legs, which thread lies below the planned breaking point. In this regard, the fixation screw can be put in place more easily if a sleeve thread is also present in the tissue-protecting sleeve, subsequent to the inside thread, at least over part of the longitudinal expanse of the tissue-protecting sleeve.

The advantages and effects mentioned above result from a sleeve remover having a corpus structured as a hollow cylinder and having a drive opening in its circumference, which corpus has a separation element at one end, on its inside circumference, which element is structured as a blade and extends over part of the circumference direction, wherein the sleeve remover has this configuration for interaction with the bone screw mentioned initially.

The characteristics and combinations of characteristics mentioned above in the description, as well as the characteristics and combinations of characteristics mentioned below in the figure description and/or characteristics and combinations of characteristics solely shown in the figures can be used not only in the combination indicated, in each instance, but rather also in other combinations or by themselves, without departing from the scope of the invention. There embodiments that are not explicitly shown in the figures or explained but are evident and can be produced from the embodiments explained, by means of separate combinations of characteristics, must also be viewed as being covered and disclosed by the invention.

Further advantages, characteristics and details of the invention are evident from the claims, the following description of preferred embodiments, and using the drawings. In this regard, the figures show:

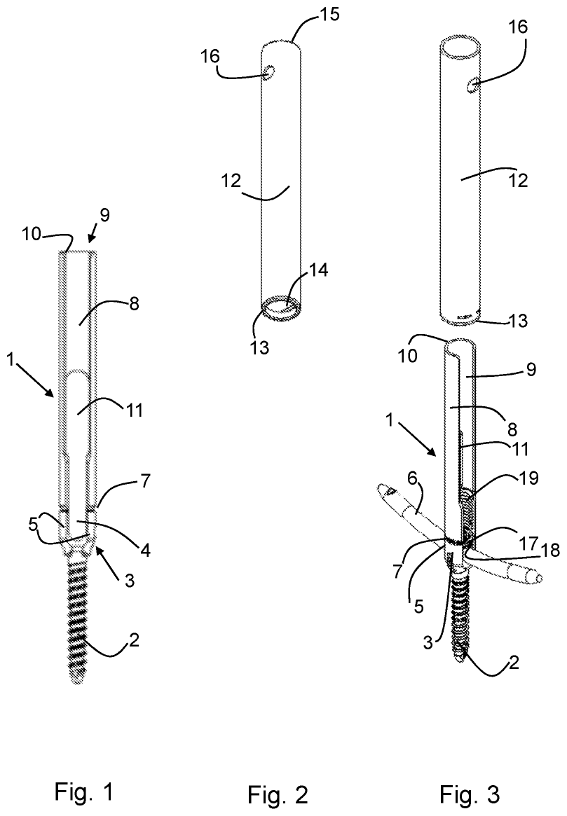

FIG. 1 a side view of a bone screw, the screw head of which, having two legs that form a receptacle, is connected with a tissue-protecting sleeve by way of a planned breaking point,

FIG. 2 a perspective representation of a sleeve remover, which has a separation element on its inside circumference surface,

FIG. 3 a perspective representation of the bone screw, used as a pedicle screw, into the receptacle of which, in the screw head, a rod is placed between the two legs and secured by means of a fixation screw, with the tissue-protecting sleeve connected with the screw head, for the removal of which sleeve the sleeve remover is made available,

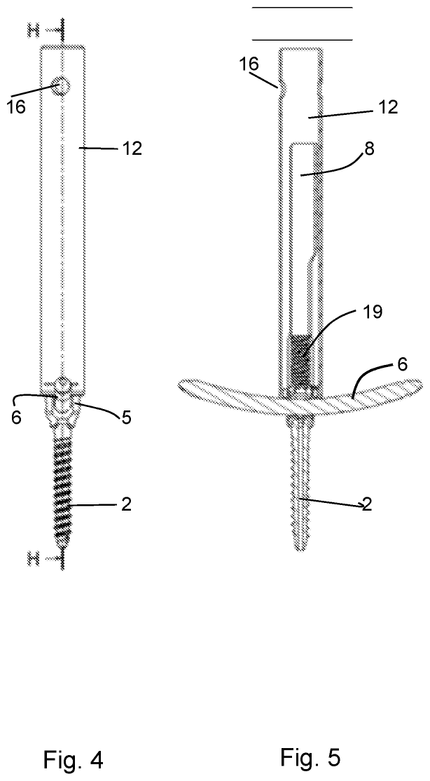

FIG. 4 a side section of the pedicle screw from FIG. 3, with the tissue-protecting sleeve and the rod, in a view onto the end face of the rod, and

FIG. 5 the section H—H from FIG. 4.

In FIG. 1, a bone screw 1 is shown in the concrete embodiment as a pedicle screw. The bone screw 1 is configured with a screw shank 2 having a thread and with a screw head 3 that has two legs 5 that lie opposite one another and form a receptacle 4 between them. A rod 6 can be laid into this receptacle 4, which rod serves for correction and stabilization of a spinal column, in that adjacent vertebra or groups of vertebrae are connected with a corresponding number of pedicle screws, by means of the rod 6.

The legs 5f, which form a tulip head, are connected, in one piece, in the exemplary embodiment shown, with a tissue-protecting sleeve 8, by way of a planned breaking point 7, which sleeve has a slot 9 that extends from its one edge all the way to the opposite edge, wherein the tissue-protecting sleeve 8 is circular in cross-section and the slot 9 extends over an arc having a length that is selected from an interval between 15° and 90°.

The tissue-protecting sleeve 8 has a stabilization bracket 10 on the edge that lies opposite the legs 5 and, on the side opposite the slot 9, an opening 11 that extends from the edge facing the leg 5, over part of the longitudinal expanse of the tissue-protecting sleeve.

FIG. 2 shows that a sleeve remover 12 that can be placed over the tissue-protecting sleeve 8 is provided, which remover has a separation element 14 on its inside circumference, in the configuration in which it is set onto the tissue-protecting sleeve 8, on its end 13 facing the legs 5, the expanse of which element, in the circumference direction, is smaller than the expanse of the slot 9 that lies in the circumference direction, so that the separation element 14, when the sleeve remover 12 is set onto the tissue-protecting sleeve 8, is introduced into the slot 9 and can be guided downward through the slot 9 all the way to the planned breaking point 7. In this regard, the separation element 14 is configured as a blade.

The sleeve remover 12 therefore has a corpus 15 structured as a hollow cylinder, which corpus carries the separation element 14 at the end 13, on its inside circumference, which element extends over part of the circumference direction. In this regard, the expanse is dimensioned in such a manner that the separation element 14 fits into the slot 9, in other words can be easily introduced into it. To exert a torque and turn the sleeve remover 12, the corpus 15 has a drive opening 16 in its circumference, wherein the drive opening 16 is arranged, in the exemplary embodiment shown, at the end facing away from the legs 5, and on the side that lies opposite the separation element 14.

FIG. 3 shows that a rod 6 can be placed in the receptacle 4 between the legs 5. The sleeve remover 12 can be pushed axially onto the tissue-protecting sleeve 8, wherein the separation element 14 is introduced into the slot 9. In this regard, the separation element 14 is at such a distance from the end 13 of the sleeve remover 12 that the separation element 14 is assigned to the planned breaking point 7 when the end 13 lies on the rod 6. If the sleeve remover 12 is now rotated by means of a pin inserted into the drive opening 16, the blade separates the tissue-protecting sleeve 8 from the legs 5 of the screw head 2 of the pedicle screw at the planned breaking point 7. In this regard, it should be noted that the planned breaking point is pressed in the direction of the screw axis, in other words it is ensured that the legs 5 cannot bend open and that a loss of clamping force of a fixation screw 17 is prevented, for which screw an inside thread 18 is formed on the inside of the legs 5, which thread lies below the planned breaking point 7 and brings about securing of the rod 6 in the receptacle 4 between the legs 5, in interaction with the fixation screw 17. In this regard, the fixation screw can be placed more easily, since a sleeve thread 19 is also present in the tissue-protecting sleeve 8, subsequent to the inside thread 18, at least over part of the longitudinal expanse of the tissue-protecting sleeve 8.

### REFERENCE SYMBOL LIST

1 bone screw
2 screw shank
3 screw head
4 receptacle
5 leg
6 rod
7 planned breaking point
8 tissue-protecting sleeve
9 slot
10 stabilization bracket
11 opening
12 sleeve remover
13 end
14 separation element
15 corpus
16 drive opening
17 fixation screw
18 inside thread
19 sleeve thread

1. A bone screw (1) having a screw shank (2) that has a thread, and having a screw head (3) that has two legs (5) that lie opposite one another and form a receptacle (4) between them, which are connected, in one piece, with a tissue-protecting sleeve (8), wherein the tissue-protecting sleeve has a slot (9) that extends from its one edge all the way to the opposite edge.

2. The bone screw (1) according to claim 1, wherein the legs (5) of the screw head (3) are connected with the tissue-protecting sleeve (8) by way of a planned breaking point (7).

3. The bone screw (1) according to claim 1, wherein the tissue-protecting sleeve (8) has a stabilization bracket (10) on the edge opposite to the legs (5).

4. The bone screw (1) according to claim 1, wherein the tissue-protecting sleeve (8) has an opening (11) on the side opposite the slot (9), which opening extends from the edge facing the leg (5), over part of the longitudinal expanse of the tissue-protecting sleeve (8).

5. The bone screw (1) according to claim 1, wherein the tissue-protecting sleeve (8) is circular in cross-section and the slot (9) extends over an arc having a length between 15° and 90°.

6. The bone screw (1) according to claim 1, wherein a sleeve remover (12) that can be placed over the tissue-protecting sleeve (8) is provided, which remover has a separation element (14) on its inside circumference, in the configuration placed onto the tissue-protecting sleeve (8), at its end (13) that faces the legs (5), the expanse of which element, which lies in the circumference direction, is smaller than the expanse of the slot (9), which lies in the circumference direction.

7. The bone screw (1) according to claim 6, wherein the separation element (14) is configured as a blade.

8. The bone screw (1) according to claim 6, wherein the sleeve remover (12) has a drive opening (16) on its circumference.

9. The bone screw (1) according to claim 8, wherein the drive opening (16) is arranged on the end that faces away from the legs (5) and/or on the side opposite the separation element (14).

10. The bone screw (1) according to claim 6, wherein a rod (6) can be placed in the receptacle (4) between the legs (5), and wherein the separation element (14) is so far away from the end (13) of the sleeve remover (12) that the separation element (14) is assigned to the planned breaking point (7) when the end (13) lies on the rod (6).

11. The bone screw (1) according to claim 2, wherein an inside thread (18) for accommodating a fixation screw (17) is formed on the inside of the legs (5), which thread lies below the planned breaking point (7).

12. A sleeve remover (12) having a corpus (15) structured as a hollow cylinder, which has a separation element (14) at one end, on its inside circumference, which element extends over part of the circumference direction.

13. The sleeve remover (12) according to claim 12, wherein the separation element (14) is configured as a blade.

14. The sleeve remover (12) according to claim 12, wherein the corpus (15) has a drive opening (16) in its circumference.
